(19) United States
(12) Patent Application Publication
(10) Pub. No.: US 2014/0276233 A1
(43) Pub. Date: Sep. 18, 2014

(54) CATHETER FORCE MEASUREMENT APPARATUS AND METHOD

(71) Applicant: CORINDUS, INC., Waltham, MA (US)

(72) Inventor: John Murphy, North Reading, MA (US)

(73) Assignee: CORINDUS, INC., Waltham, MA (US)

(21) Appl. No.: 14/220,717

(22) Filed: Mar. 20, 2014

Related U.S. Application Data
(63) Continuation of application No. PCT/US2012/056229, filed on Sep. 20, 2012.
(60) Provisional application No. 61/536,949, filed on Sep. 20, 2011.

(51) Int. Cl. A61B 19/00

(52) U.S. Cl. CPC A61B 19/46; A61B 19/2203; A61B 2019/464; A61B 2019/2211
USPC 600/587; 606/130

(57) ABSTRACT

A force measurement apparatus includes a housing having a track with a curved guide wall having a convex shape configured to guide a portion of a guide wire. A sensor is proximate the first guide wall in a first position, the sensor senses movement of a portion of the guide wire moving from a first position proximate the curved guide wall to a second position distal the first guide wall in a direction perpendicular to the longitudinal axis of the guide wire.

### CROSS-REFERENCE TO RELATED APPLICATIONS

This application claims the benefit of U.S. Provisional Application No. 61/536,949, filed Sep. 20, 2011, and PCT/US2012/056229 entitled CATHETER FORCE MEASUREMENT APPARATUS AND METHOD both of which are incorporated herein by reference in their entirety.

### BACKGROUND

The present invention relates generally to the field of catheter systems for performing diagnostic and/or intervention procedures. The present invention relates specifically to an apparatus and method for measuring the force applied to a free end of a guide wire and/or working catheter.

Vascular disease, and in particular cardiovascular disease, may be treated in a variety of ways. Surgery, such as cardiac bypass surgery, is one method for treating cardiovascular disease. However, under certain circumstances, vascular disease may be treated with a catheter based intervention procedure, such as angioplasty. Catheter based intervention procedures are generally considered less invasive than surgery. If a patient shows symptoms indicative of cardiovascular disease, an image of the patient's heart may be taken to aid in the diagnosis of the patient's disease and to determine an appropriate course of treatment. For certain disease types, such as atherosclerosis, the image of the patient's heart may show a lesion that is blocking one or more coronary arteries. Following the diagnostic procedure, the patient may undergo a catheter based intervention procedure. During one type of intervention procedure, a catheter is inserted into the patient's femoral artery and moved through the patient's arterial system until the catheter reaches the site of the lesion. In some procedures, the catheter is equipped with a balloon or a stent that when deployed at the site of a lesion allows for increased blood flow through the portion of the coronary artery that is affected by the lesion. In addition to cardiovascular disease, other diseases (e.g., hypertension, etc.) may be treated using catheterization procedures.

### SUMMARY

One embodiment of the invention relates to a force measurement apparatus including a housing having a track with a curved guide wall having a convex shape configured to guide a portion of a guide wire. A sensor is proximate the first guide wall in a first position, the sensor senses movement of a portion of the guide wire moving from a first position proximate the curved guide wall to a second position distal the first guide wall in a direction perpendicular to the longitudinal axis of the guide wire.

Another embodiment of the invention relates to a robotic catheter including a housing and a linear drive mechanism supported by the housing and configured to engage and to impart linear movement to a guide wire along a longitudinal axis of the guide wire. A track includes a curved guide wall configured to guide a portion of the guide wire in an arcuate path and an open region allowing a portion of the guide wire to move into the open region in response to a force being applied to a free end of the guide wire. A sensor is proximate the first guide wall in a first position, the sensor senses movement of a portion of the guide wire moving from a first position proximate the curved guide wall to a second position distal the first guide wall in a direction perpendicular to the longitudinal axis of the guide wire.

Another embodiment of the invention relates to a method for measuring the force on a guide wire and/or working catheter, including providing a channel having a first linear section with a wall on each side of the guide wire in a direction perpendicular to the movement of the guide wire. A second curved convex section is provided having a single wall and an open region in a direction perpendicular to the direction of travel. A portion of the guide wire is permitted to move from the curved convex section toward the open region in response to a force applied to a free end of the guide wire. A sensor is operatively connected in the open region proximate the curved convex to measure movement of the guide wire away from the curved convex section toward the open region. A signal is provided to a control station of the related to the amount movement of the guide wire from the a curved convex section toward the open region.

Alternative exemplary embodiments relate to other features and combinations of features as may be generally recited in the claims.

### BRIEF DESCRIPTION OF THE DRAWINGS

This application will become more fully understood from the following detailed description, taken in conjunction with the accompanying figures, wherein like reference numerals refer to like elements in which:

FIG. 1 is a perspective view of a catheter procedure system according to an exemplary embodiment;

FIG. 2 is a block diagram of a catheter procedure system according to an exemplary embodiment;

FIG. 3 is a perspective view of a bedside system showing an embodiment of a cassette prior to being attached to a motor drive base;

FIG. 4 is a perspective view of a bedside system showing the cassette of FIG. 3 following attachment to the motor drive base;

FIG. 5 is a rear perspective view of a cassette according to an exemplary embodiment;

FIG. 6 is an enlarged perspective view of a guide catheter support in a first position according to an exemplary embodiment;

FIG. 7 is an enlarged perspective view of the guide catheter support of FIG. 6 in a second position according to an exemplary embodiment;

FIG. 8 is a perspective view of a cassette in the “loading” configuration;

FIG. 9 is a perspective view of a cassette in the “loaded” or “use” configuration;

FIG. 10 is an exploded perspective view of an axial drive assembly of a cassette;

FIG. 11 is a bottom perspective view of a cassette showing the base plate removed;

FIG. 12 is a top view showing the axial drive assembly in the “disengaged” position;

FIG. 13 is a top view showing the axial drive assembly in the “engaged” position;

FIG. 14 is a top perspective view of a rotational drive assembly of a cassette showing the engagement structure in broken lines beneath the chassis;

FIG. 15 is a top perspective view of a rotational drive assembly with the chassis shown in broken lines;

FIG. 16 is a top view of the rotational drive assembly in the “engaged” position;

FIG. 17 is a top view of the rotational drive assembly in the “disengaged” position; and

FIG. 18 is a rear perspective view of a cassette according to an exemplary embodiment.

FIG. 19 is a top perspective view of a catheter force measurement device for use with a catheter drive mechanism.

FIG. 20 is a top view of the catheter force measurement module in a first position and a second position shown in dashed lines.

FIG. 21 is a cross-sectional view taken generally along lines 21-21 of FIG. 20 when the guide wire is in a neutral non-stressed position.

FIG. 22 is a cross-sectional view taken generally along lines 21-21 of FIG. 20 when the guide wire is a flexed stressed position.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring to FIG. 1, a catheter procedure system 10 is shown. Catheter procedure system 10 may be used to perform catheter based medical procedures (e.g., percutaneous intervention procedures). Percutaneous intervention procedures may include diagnostic catheterization procedures during which one or more catheters are used to aid in the diagnosis of a patient's disease. For example, during one embodiment of a catheter based diagnostic procedure, a contrast media is injected into one or more coronary arteries through a catheter and an image of the patient's heart is taken. Percutaneous intervention procedures may also include catheter based therapeutic procedures (e.g., balloon angioplasty, stent placement, treatment of peripheral vascular disease, etc.) during which a catheter is used to treat a disease. It should be noted, however, that one skilled in the art would recognize that certain specific percutaneous intervention devices or components (e.g., type of guide wire, type of catheter, etc.) will be selected based on the type of procedure that is to be preformed. Catheter procedure system 10 is capable of performing any number of catheter based medical procedures with minor adjustments to accommodate the specific percutaneous devices to be used in the procedure. In particular, while the embodiments of catheter procedure system 10 described herein are explained primarily in relation to the diagnosis and/or treatment of coronary disease, catheter procedure system 10 may be used to diagnose and/or treat any type of disease or condition amenable to diagnosis and/or treatment via a catheter based procedure.

Catheter procedure system 10 includes lab unit 11 and workstation 14. Catheter procedure system 10 includes a robotic catheter system, such as bedside system 12, located within lab unit 11 adjacent patient 21. Generally, bedside system 12 may be equipped with the appropriate percutaneous devices (e.g., guide wires, guide catheters, working catheters, catheter balloons, stents, diagnostic catheters, etc.) or other components (e.g., contrast media, medicine, etc.) to allow the user to perform a catheter based medical procedure. A robotic catheter system, such as bedside system 12, may be any system configured to allow a user to perform a catheter based medical procedure via a robotic system by operating various controls such as the controls located at workstation 14. Bedside system 12 may include any number and/or combination of components to provide bedside system 12 with the functionality described herein. Bedside system 12 may include a cassette 56 coupled to a base 19, and cassette 56 may include a housing 22 that supports the various components of the cassette. One particular embodiment of a cassette (shown as cassette 300) is described below in relation to FIGS. 3-18.

In one embodiment, bedside system 12 may be equipped to perform a catheter based diagnostic procedure. In this embodiment, bedside system 12 may be equipped with one or more of a variety of catheters for the delivery of contrast media to the coronary arteries. In one embodiment, bedside system 12 may be equipped with a first catheter shaped to deliver contrast media to the coronary arteries on the left side of the heart, a second catheter shaped to deliver contrast media to the coronary arteries on the right side of the heart, and a third catheter shaped to deliver contrast media into the chambers of the heart.

In another embodiment, bedside system 12 may be equipped to perform a catheter based therapeutic procedure. In this embodiment, bedside system 12 may be equipped with a guide catheter, a guide wire, and a working catheter (e.g., a balloon catheter, a stent delivery catheter, ablation catheter, etc.). In one embodiment, the working catheter may be an over-the-wire working catheter that includes a central lumen that is threaded over the guide wire during a procedure. In another embodiment, the working catheter includes a secondary lumen that is separate from the central lumen of the working catheter, and the secondary lumen is threaded over the guide wire during a procedure. In another embodiment, bedside system 12 may be equipped with an intravascular ultrasound (IVUS) catheter. In another embodiment, any of the percutaneous devices of bedside system 12 may be equipped with positional sensors that indicate the position of the component within the body.

Bedside system 12 is in communication with workstation 14, allowing signals generated by the user inputs and control system of workstation 14 to be transmitted to bedside system 12 to control the various functions of beside system 12. Bedside system 12 also may provide feedback signals (e.g., operating conditions, warning signals, error codes, etc.) to workstation 14. Bedside system 12 may be connected to workstation 14 via a communication link 38 that may be a wireless connection, cable connectors, or any other means capable of allowing communication to occur between workstation 14 and beside system 12.

Workstation 14 includes a user interface 30 configured to receive user inputs to operate various components or systems of catheter procedure system 10. User interface 30 includes controls 16. Controls 16 allow the user to control bedside system 12 to perform a catheter based medical procedure. For example, controls 16 may be configured to cause bedside system 12 to perform various tasks using the various percutaneous devices with which bedside system 12 may be equipped (e.g., to advance, retract, or rotate a guide wire, advance, retract, or rotate a working catheter, advance, retract, or rotate a guide catheter, inflate or deflate a balloon located on a catheter, position and/or deploy a stent, inject contrast media into a catheter, inject medicine into a catheter, or to perform any other function that may be performed as part of a catheter based medical procedure, etc.). In some embodiments, one or more of the percutaneous intervention devices may be steerable, and controls 16 may be configured to allow a user to steer one or more steerable percutaneous device. In one such embodiment, bedside system 12 may be equipped with a steerable guide catheter, and controls 16 may also be configured to allow the user located at remote workstation 14 to control the bending of the distal tip of a steerable guide catheter.

In one embodiment, controls 16 include a touch screen 18, a dedicated guide catheter control 29, a dedicated guide wire control 23, and a dedicated working catheter control 25. In this embodiment, guide wire control 23 is a joystick configured to advance, retract, or rotate a guide wire, working catheter control 25 is a joystick configured to advance, retract, or rotate a working catheter, and guide catheter control 29 is a joystick configured to advance, retract, or rotate a guide catheter. In addition, touch screen 18 may display one or more icons (such as icons 162, 164, and 166) that control movement of one or more percutaneous devices via bedside system 12. Controls 16 may also include a balloon or stent control that is configured to inflate or deflate a balloon and/or a stent. Each of the controls may include one or more buttons, joysticks, touch screens, etc., that may be desirable to control the particular component to which the control is dedicated.

Controls 16 may include an emergency stop button 31 and a multiplier button 33. When emergency stop button 31 is pushed a relay is triggered to cut the power supply to bedside system 12. Multiplier button 33 acts to increase or decrease the speed at which the associated component is moved in response to a manipulation of guide catheter control 29, guide wire control 23, and working catheter control 25. For example, if operation of guide wire control 23 advances the guide wire at a rate of 1 mm/sec, pushing multiplier button 33 may cause the operation of guide wire control 23 to advance the guide wire at a rate of 2 mm/sec. Multiplier button 33 may be a toggle allowing the multiplier effect to be toggled on and off. In another embodiment, multiplier button 33 must be held down by the user to increase the speed of a component during operation of controls 16.

User interface 30 may include a first monitor 26 and a second monitor 28. First monitor 26 and second monitor 28 may be configured to display information or patient-specific data to the user located at workstation 14. For example, first monitor 26 and second monitor 28 may be configured to display image data (e.g., x-ray images, MRI images, CT images, ultrasound images, etc.), hemodynamic data (e.g., blood pressure, heart rate, etc.), patient record information (e.g., medical history, age, weight, etc.). In one embodiment, monitors 26 and/or 28 may be configured to display an image of a portion of the patient (e.g., the patient's heart) at one or more magnification levels. In addition, first monitor 26 and second monitor 28 may be configured to display procedure specific information (e.g., duration of procedure, catheter or guide wire position, volume of medicine or contrast agent delivered, etc.). Monitor 26 and monitor 28 may be configured to display information regarding the position and/or bend of the distal tip of a steerable guide catheter. Further, monitor 26 and monitor 28 may be configured to display information to provide the functionalities associated with the various modules of controller 40 discussed below. In another embodiment, user interface 30 includes a single screen of sufficient size to display one or more of the display components and/or touch screen components discussed herein.

Catheter procedure system 10 also includes an imaging system 32 located within lab unit 11. Imaging system 32 may be any medical imaging system that may be used in conjunction with a catheter based medical procedure (e.g., non-digital x-ray, digital x-ray, CT, MRI, ultrasound, etc.). In an exemplary embodiment, imaging system 32 is a digital x-ray imaging device that is in communication with workstation 14. Referring to FIG. 1, imaging system 32 may include a C-arm that allows imaging system 32 to partially or completely rotate around patient 21 in order to obtain images at different angular positions relative to patient 21 (e.g., sagital views, caudal views, cranio-caudal views, etc.).

Imaging system 32 is configured to take x-ray images of the appropriate area of patient 21 during a particular procedure. For example, imaging system 32 may be configured to take one or more x-ray images of the heart to diagnose a heart condition. Imaging system 32 may also be configured to take one or more x-ray images during a catheter based medical procedure (e.g., real-time images) to assist the user of workstation 14 to properly position a guide wire, guide catheter, working catheter, stent, etc. during the procedure. The image or images may be displayed on first monitor 26 and/or second monitor 28.

In addition, the user of workstation 14 may be able to control the angular position of imaging system 32 relative to the patient to obtain and display various views of the patient's heart on first monitor 26 and/or second monitor 28. Displaying different views at different portions of the procedure may aid the user of workstation 14 to properly move and position the percutaneous devices within the 3D geometry of the patient's heart. In an exemplary embodiment, imaging system 32 may be any 3D imaging modality of the past, present, or future, such as an x-ray based computed tomography (CT) imaging device, a magnetic resonance imaging device, a 3D ultrasound imaging device, etc. In this embodiment, the image of the patient's heart that is displayed during a procedure may be a 3D image. In addition, controls 16 may also be configured to allow the user positioned at workstation 14 to control various functions of imaging system 32 (e.g., image capture, magnification, collimation, c-arm positioning, etc.).

Referring to FIG. 2, a block diagram of catheter procedure system 10 is shown according to an exemplary embodiment. Catheter procedure system 10 may include a control system, such as controller 40. Controller 40 may be part of workstation 14. Controller 40 may generally be an electronic control unit suitable to provide catheter procedure system 10 with the various functionalities described herein. For example, controller 40 may be an embedded system, a dedicated circuit, a general purpose system programmed with the functionality described herein, etc. Controller 40 is in communication with one or more bedside systems 12, controls 16, monitors 26 and 28, imaging system 32, and patient sensors 35 (e.g., electrocardiogram (“ECG”) devices, electroencephalogram (“EEG”) devices, blood pressure monitors, temperature monitors, heart rate monitors, respiratory monitors, etc.). In various embodiments, controller 40 is configured to generate control signals based on the user's interaction with controls 16 and/or based upon information accessible to controller 40 such that a medical procedure may be preformed using catheter procedure system 10. In addition, controller 40 may be in communication with a hospital data management system or hospital network 34, and one or more additional output devices 36 (e.g., printer, disk drive, cd/dvd writer, etc.).

Communication between the various components of catheter procedure system 10 may be accomplished via communication links 38. Communication links 38 may be dedicated wires or wireless connections. Communication links 38 may also represent communication over a network. Catheter procedure system 10 may be connected or configured to include any other systems and/or devices not explicitly shown. For example, catheter procedure system 10 may include IVUS systems, image processing engines, data storage and archive systems, automatic balloon and/or stent inflation systems, medicine tracking and/or logging systems, user logs, encryption systems, systems to restrict access or use of catheter procedure system 10, robotic catheter systems of the past, present, or future, etc.

Referring now to FIGS. 3 through 18, an exemplary embodiment of a cassette for use with a robotic catheter system is shown. Cassette 300 may be equipped with a guide wire 301 and a working catheter 303 to allow a user to perform a catheterization procedure utilizing cassette 300. In this embodiment, bedside system 12 includes a cassette 300 configured to be mounted to a motor drive base 302. FIG. 3 shows a bottom perspective view of cassette 300 prior to mounting to motor drive base 302. Motor drive base 302 includes a first capstan 304, a second capstan 306, and a third capstan 308, and cassette 300 includes a first capstan socket 310, a second capstan socket 312, and a third capstan socket 314. Cassette 300 includes a housing 316, and housing 316 includes a base plate 318.

Each of the capstan sockets is configured to receive one of the capstans of motor drive base 302. In the embodiment shown, base plate 318 includes a hole or aperture aligned with each of the capstan sockets 310, 312, and 314 to allow each capstan to engage with the appropriate capstan socket. The engagement between the capstans and capstan sockets allows the transfer of energy (e.g., rotational movement) generated by one or more actuators (e.g., motors) located within motor drive base 302 to each of the drive mechanisms (discussed below) within cassette 300. In one embodiment, a single actuator provides energy to each of the drive mechanisms. In another embodiment, there is an actuator that drives capstan 304, an actuator that drives capstan 306, and an actuator that drives capstan 308. Further, the positioning of the capstans and capstan sockets helps the user to align cassette 300 relative to motor drive base 302 by allowing cassette 300 to be mounted to motor drive base 302 only when all three capstan sockets are aligned with the proper capstan.

In one embodiment, the motors that drive capstans 304, 306, and 308 are located within motor drive base 302. In another embodiment, the motors that drive capstans 304, 306, and 308 may be located outside of base 302 connected to cassette 300 via an appropriate transmission device (e.g., shaft, cable, etc.). In yet another embodiment, cassette 300 includes motors located within the housing of cassette 300. In another embodiment, cassette 300 does not include capstan sockets 310, 312, and 314, but includes an alternative mechanism for transferring energy (e.g., rotational motion) from an actuator external to the cassette to each of the cassette drive mechanisms. For example, rotational movement may be transferred to the drive mechanisms of cassette 300 via alternating or rotating magnets or magnetic fields located within motor drive base 302.

In the embodiment shown, cassette 300 also includes a guide catheter support 311 that supports guide catheter 317 at a position spaced from cassette 300. As shown, guide catheter support 311 is attached to cassette 300 by a rod 313. Rod 313 and guide catheter support 311 are strong enough to support guide catheter 317 without buckling. Guide catheter support 311 supports guide catheter 317 at a position spaced from the cassette, between the patient and the cassette to prevent buckling, bending, etc. of the portion of guide catheter 317 between the cassette and the patient.

Referring to FIG. 4, cassette 300 is shown mounted to motor drive base 302. As shown in FIG. 4, cassette 300 includes an outer cassette cover 320 that may be attached to housing 316. When attached to housing 316, outer cassette cover 320 is positioned over and covers each of the drive mechanisms of cassette 300. By covering the drive assemblies of cassette 300, outer cassette cover 320 acts to prevent accidental contact with the drive mechanisms of cassette 300 while in use.

In various embodiments, cassette 300 may be configured to provide for secure (e.g., stabile, rigid, locked, etc.) attachment of cassette 300 to motor drive base 302. In various embodiments, motor drive base 302 may impart generally upwardly directed forces onto cassette 300 as the various components of motor drive base 302 engage with cassette 300 to provide the functionalities discussed herein. Cassette 300 may be configured to attach or couple to motor drive base 302 in a way that ensures that cassette 300 remains coupled to motor drive base 302 despite the application of upward forces during use. In various embodiments, cassette 300 may include one or more structures extending from the housing of the cassette that are configured to be received by or within one or more corresponding mating structures on motor drive base 302 in a manner that will resist or prevent upward motion of cassette 300 away from motor drive base 302.

Referring to FIG. 5, a rear perspective view of cassette 300 is shown with outer cassette cover 320 attached to housing 316. In the embodiment shown in FIG. 5, cassette 300 may include one or more arms or tabs, shown as mounting tabs 600, extending substantially perpendicular to the plane defined by the side wall of housing 316. In the specific embodiment shown, cassette 300 includes two tabs 600, one located toward the rear of cassette 300 and one located toward the front of cassette 300. Mounting tabs 600 each include an upper surface 604 and a lower surface 606. In the embodiment shown, upper surface 604 and lower surface 606 are substantially planar surfaces. Upper surface 604 is substantially parallel to lower surface 606, and both are substantially parallel to the lower surface of base plate 318. Mounting tabs 600 are positioned along the lower or bottom edge of housing 316 such that lower surface 606 of each tab and the lower surface of base plate 318 form a substantially planar lower surface of cassette 300.

Mounting tabs 600 are configured to engage or mate with a receiving structure on motor drive base 302 to provide resistance to upward forces generated by motor drive base 302 to help ensure that cassette 300 remains mounted to motor drive base 302 during application of such forces. In one embodiment, motor drive base 302 includes a pair of brackets 602 shown in FIG. 3. When cassette 300 is mounted to motor drive base 302, the mounting tabs 600 are received within brackets 602 such that upper surfaces 604 of the mounting tabs 600 are in contact with the lower surfaces of brackets 602. The contact between upper surfaces 604 and brackets 602 tends to resist upward movement of cassette 300 that may otherwise occur without this engagement. The resistance of upward movement helps to ensure proper functioning of cassette 300 by helping to ensure that the proper engagement between cassette 300 and motor drive base 302 is maintained during a procedure.

While FIG. 3 shows the receiving structure of motor drive base 302 as a generally u-shaped bracket, other receiving structures may be utilized. For example, in one embodiment, the receiving structure may include a plurality of recesses formed in the upper surface of motor drive base 302 configured to receive mounting tabs 600. In another embodiment, motor drive base 302 may include one or more arms that are moveable between and clamped and unclamped positions, and in the clamped position, the moveable arm engages upper surface 604 of each mounting tab 600 such that upward movement of cassette 300 may be resisted.

Referring to FIG. 6 and FIG. 7, guide catheter support 311 is shown according to an exemplary embodiment. Guide catheter support 311 is coupled to the distal end of rod 313, and, as shown in FIG. 3, the proximal end of rod 313 is coupled to housing 316 of cassette 300. Guide catheter support 311 supports guide catheter 317 at a position spaced from cassette 300. Rod 313 and guide catheter support 311 are strong enough to support guide catheter 317 without buckling. Guide catheter support 311 supports guide catheter 317 to prevent buckling, bending, etc. of the portion of guide catheter 317 between the cassette and the patient.

Guide catheter support 311 includes a body 620. Body 620 defines a longitudinal axis that, in the embodiment shown, is substantially perpendicular to the longitudinal axis of rod 313. Body 620 includes a first end 622. A guide catheter engaging structure, shown as clamp 624, is located adjacent to first end 622 of body 620. Clamp 624 is configured to engage guide catheter 317 such that guide catheter 317 is held in position (i.e., prevented from moving) relative to guide catheter support 311 and/or cassette 300.

In the embodiment shown, clamp 624 includes a pivoting member 626 and a biasing element, shown as spring 628, engaged between pivoting member 626 and body 620. Spring 628 biases clamp 624 into engagement with guide catheter 317, as shown in FIGS. 6 and 7. In the embodiment shown, pivoting member 626 includes an engagement surface, shown as curved recess 630, and body 620 includes an engagement surface, shown as curved recess 632, that is opposed to recess 630. Guide catheter 317 is engaged between a lower surface of pivoting member 626 and an upper surface of body 620 such that guide catheter 317 is received within curved recesses 630 and 632. As shown, in FIGS. 6 and 7, curved recesses 630 and 632 are located between first end 622 and the center point of body 620 (and consequently between first end 622 and second end 636), and further, spring 628 is located between first end 622 and recesses 630 and 632.

To move clamp 624 from the engaged position shown in FIGS. 6 and 7, to the open position (not shown), a force, such as a force applied by a user's thumb, is applied to the outer end 634 of pivoting member 626 causing compression of spring 628. With clamp 624 in the open position, guide catheter 317 is placed within recess 632 of body 620. When the force is removed from outer end 634, spring 628 expands causing clamp 624 to move to the closed position engaging guide catheter 317.

Located at the second end 636 of body 620 is a rotation joint, shown as rotatable joint 638, coupling guide catheter support 311 to rod 313. As can be seen from a comparison of FIGS. 6 and 7, rotatable joint 638 allows body 620 and clamp 624 of guide catheter support 311 to rotate about the longitudinal axis of body 620. In FIG. 6, arrow line 640 indicates the direction of rotation provided by rotatable joint 638. In the embodiment shown, body 620 of guide catheter support 311 rotates about an axis substantially perpendicular to a longitudinal axis defined by rod 313.

As illustrated in FIGS. 6 and 7, rotatable joint 638 allows guide catheter support 311 to accommodate and engage guide catheters 317 positioned at a variety of angles. During a catheterization procedure, the angle at which a guide catheter is positioned may vary due to a number of factors (e.g., size of the patient, location of entry incision, type of guide catheter used, etc.). Thus, rotatable joint 638 allows guide catheter support 311 to accommodate a wider range of guide catheter positions than if guide catheter support 311 did not include a rotatable connection to rod 313. In one embodiment, guide catheter support 311 may be rotated about the longitudinal axis of guide catheter support 311 via rotatable joint 638 such that the engagement surfaces are able to engage the guide catheter 317 at a plurality of angular positions relative to the patient's body. Specifically, guide catheter support 311 may be rotated such that the engagement surfaces are substantially parallel to the longitudinal axis of guide catheter 317 such that the engagement surfaces engage the outer surface of the guide catheter when clamp 624 is moved to the closed, engaged position.

In one embodiment, guide catheter support 311 may be rotated about rotatable joint 638 manually. In another embodiment, guide catheter support 311 or cassette 300 may include an actuator (e.g., a step motor, etc.) that controls the rotational position of guide catheter support 311. In this embodiment, controls 16 may include a control or user input (e.g., a dial, joystick, touch screen icon, etc.) associated with the guide catheter support 311 such that a user located at workstation 14 may control or change the rotational position of guide catheter support 311 by manipulating the control located at workstation 14.

Referring to FIG. 8, cassette 300 is shown in the “loading” configuration with outer cassette cover 320 removed. Cassette 300 includes a y-connector support assembly 322, an axial drive assembly 324, and a rotational drive assembly 326. Generally, the various portions of cassette 300 are placed in the loading configuration to allow the user to load or install a guide wire and/or working catheter into cassette 300. Further, in the exemplary embodiment shown, y-connector support assembly 322 is located in front of axial drive assembly 324, and axial drive assembly 324 is located in front of rotational drive assembly 326 within cassette 300.

Y-connector support assembly 322 includes a chassis 328 and a y-connector restraint 330. Base plate 318 includes a support arm 332 that supports y-connector support assembly 322. Chassis 328 is coupled to the front of support arm 332 via pin connection 334.

A central groove or depression 336 extends the length of chassis 328. Y-connector 338 rests within central groove 336 of chassis 328. Y-connector 338 includes a first leg 340, a second leg 342, and a third leg 344. First leg 340 is configured to attach to a guide catheter such that the central lumen of the y-connector is in fluid communication with the central lumen of the guide catheter. Second leg 342 is angled away from the longitudinal axis of y-connector 338. Second leg 342 of y-connector 338 allows introduction of a contrast agent or medicine into the lumen of the guide catheter. A one way valve prohibits bodily fluid from exiting second leg 342. Third leg 344 extends away from the guide catheter toward axial drive assembly 324. In use, guide wire 301 and working catheter 303 are inserted into third leg 344 of y-connector 338 via opening 346 and may be advanced through y-connector 338 into the lumen of the guide catheter. The third leg also includes a one way valve that permits insertion and removal of the working catheter and guide wire but prohibits bodily fluids from exiting third leg 344.

Chassis 328 is rotatable about an axis defined by pin connection 334 to allow chassis 328 to be placed in the “loading position” shown in FIG. 8. In the loading position, chassis 328 is positioned at about a 45 degree angle, shown by angle line 315, relative to support arm 332. Chassis 328 is moved to the “loading position” to provide easier access to opening 346 of the third leg 344 allowing the user to feed guide wire 301 and working catheter 303 into y-connector 338.

Y-connector support assembly 322 includes y-connector restraint 330. Y-connector restraint 330 is configured to releasably engage y-connector 338. In the engaged position shown in FIG. 8, engagement arm 348 of y-connector restraint 330 engages or presses y-connector 338 into central groove 336 to securely hold y-connector 338. Y-connector restraint 330 may be moved to a disengaged position to release y-connector 338 from chassis 328.

Cassette 300 also includes an axial drive assembly 324. Axial drive assembly 324 includes a first axial drive mechanism, shown as guide wire axial drive mechanism 350, and a second axial drive mechanism, shown as working catheter axial drive mechanism 352. Axial drive assembly 324 also includes a top deck 354, a cover 356, and a latch or handle 358.

Generally, guide wire axial drive mechanism 350 is configured to releasably engage and drive (e.g., to impart motion to) guide wire 301 along its longitudinal axis. In this manner, guide wire axial drive mechanism 350 provides for advancement and/or retraction of guide wire 301. Working catheter axial drive mechanism 352 is configured to releasably engage and drive (e.g., to impart motion to) working catheter 303 along its longitudinal axis. In this manner, working catheter axial drive mechanism 352 provides for advancement and/or retraction of working catheter 303.

Top deck 354 is mounted to a central portion 360 of base plate 318. Top deck 354 includes a guide wire channel 364 and a working catheter drive channel 366. Guide wire channel 364 is positioned generally perpendicular to the top surface of top deck 354 and runs the length of top deck 354 in the longitudinal direction. Working catheter drive channel 366 is positioned generally perpendicular to the top surface of top deck 354 and is located at an angle relative to guide wire channel 364. A plurality of tabs 368 extend vertically from the top surface of top deck 354 along guide wire channel 364.

In FIG. 8, cover 356 is shown in the open position. Handle 358 is moved to a position generally parallel to the longitudinal axis of cassette 300 to allow cover 356 to move to the open position. Cover 356 is mounted to top deck 354 via hinges 370. Cassette 300 includes a restraint structure that acts to restrain movement of the guide wire when cover 356 is in the closed position. As shown, the restraint structure includes a plurality of tabs 372 extending from the lower surface of cover 356. Tabs 372 are positioned such that when cover 356 is closed, tabs 372 are positioned within a portion of guide wire channel 364 between tabs 368 such that tabs 372 restrain movement of guide wire 301 in a vertical direction (i.e., restrains movement of the guide wire in a direction perpendicular to the top surface of top deck 354).

When cover 356 is in the open position, both guide wire axial drive mechanism 350 and working catheter axial drive mechanism 352 are exposed allowing the user to load cassette 300 with a guide wire and working catheter. With cover 356 open, guide wire 301 is loaded into axial drive assembly 324 by placing the guide wire into guide wire channel 364. Tabs 368 facilitate the placement of guide wire 301 by aiding the user in aligning the guide wire with guide wire channel 364. In addition, working catheter 303 is loaded into axial drive assembly 324 by placing the working catheter into working catheter drive channel 366. As will be described in more detail below, once the guide wire and working catheter are positioned within guide wire channel 364 and working catheter drive channel 366, respectively, engagement surfaces of guide wire axial drive mechanism 350 and working catheter axial drive mechanism 352 are brought into engagement with the guide wire and working catheter respectively.

Both top deck 354 and central portion 360 of base plate 318 are shaped to define a recess 374. Working catheter drive channel 366 includes an opening 376 located within recess 374. Recess 374 allows opening 376 to be closer to y-connector 338 and also closer to the entry incision in the patient allowing working catheter 303 to be advanced farther into the patient's vascular system than if opening 376 were located further away from y-connector 338 or the entry incision. As can be seen in FIG. 4, working catheter 303 includes a hub 305 at its proximal end that is too large to fit through opening 376. Thus, the closer that opening 376 is to y-connector 338 and to the entry incision the further working catheter 303 can be advanced into the patient's vascular system.

In various embodiments, cassette 300 may be configured to facilitate the performance of a catheter-based medical procedure with more than one working catheter device. For example, a procedure using cassette 300 may be performed using a first working catheter and second working catheter. In one embodiment, cassette 300 may include a third channel, shown as secondary channel 650, configured to receive and hold a working catheter when the working catheter is not positioned within working catheter drive channel 366. In contrast to channels 364 and 366, secondary channel 650 is not a channel associated with a drive mechanism and does not include a structure to engage and to impart motion to the catheter device while the catheter device is located within secondary channel 650.

Referring to the exemplary embodiment shown in FIG. 8, cassette 300 includes secondary channel 650 formed in top deck 354 of axial drive assembly 324. Secondary channel 650 is located in front of working catheter drive channel 366, and, specifically, in the embodiment shown, secondary channel 650 is located between y-connector support assembly 322 and working catheter drive channel 366. As explained in greater detail below regarding FIG. 9, secondary channel 650 provides a storage or holding location for a second working catheter device, when a different working catheter device is engaged within working catheter drive channel 366.

Like working catheter drive channel 366, secondary channel 650 is positioned generally perpendicular to the top surface of top deck 354, intersects guide wire channel 364 near the front end of guide wire channel 364 and is located at an angle relative to guide wire channel 364. Secondary channel 650 includes an opening 652 located through the sidewall of the housing of cassette 300. In the embodiment shown, opening 652 is located in front of recess 374 and also in front of opening 376 of working catheter drive channel 366. In the embodiment shown in FIG. 8, secondary channel 650 is curved, and, in another embodiment, secondary channel 650 may be a substantially straight channel.

Referring to FIG. 8, cassette 300 may include a series of additional restraint structures, shown as tab 654, tab 656 and tab 658. Tab 654, tab 656 and tab 658 extend from the lower surface of cover 356. As indicated by the dot-dash lines, when cover 356 is moved to the closed position, tab 654 is positioned within a portion of secondary channel 650, and tabs 656 and 658 are located within portions of working catheter drive channel 366. Tab 654 acts to restrain movement of a working catheter within secondary channel 650 in the vertical direction (i.e., restrains movement of the working catheter in a direction perpendicular to the top surface of top deck 354). Tab 656 and tab 658 act to restrain movement of a working catheter within working catheter drive channel 366 in the vertical direction (i.e., restrains movement of the working catheter in a direction perpendicular to the top surface of top deck 354). In the embodiment shown, tab 656 is received near the front end of working catheter drive channel 366 (i.e., the portion of working catheter drive channel 366 adjacent to guide wire channel 364), and tab 658 is received near the rear end of working catheter drive channel 366 (i.e., the portion of working catheter drive channel 366 adjacent opening 376).

Cassette 300 also includes a rotational drive assembly 326. Rotational drive assembly 326 includes a rotational drive mechanism, shown as guide wire rotational drive mechanism 380, a cover 384, and a journal 388. Guide wire rotational drive mechanism 380 includes a chassis 382 and an engagement structure 386. Rotational drive assembly 326 is configured to cause guide wire 301 to rotate about its longitudinal axis. Engagement structure 386 is configured to releasably engage guide wire 301 and to apply sufficient force to guide wire 301 such that guide wire 301 is allowed to rotate about its longitudinal axis while permitting guide wire 301 to be moved axially by guide wire axial drive mechanism 350.

In the embodiment shown, rotational drive assembly 326 is supported within housing 316 such that rotation drive assembly 326 is permitted to rotate within housing 316. Engagement structure 386 applies sufficient force to guide wire 301 that the rotation of rotation drive assembly 326 causes guide wire 301 to rotate about its longitudinal axis as rotational drive assembly 326 rotates.

Chassis 382 includes a guide wire channel 390. Guide wire channel 390 is positioned generally perpendicular to the top surface of chassis 382 and runs the length of chassis 382 in the longitudinal direction. A plurality of tabs 392 extend vertically from the top surface of chassis 382 along guide wire channel 390. In FIG. 8, cover 384 is shown in the open position. Cover 384 is mounted to chassis 382 via hinge 394. Cassette 300 includes a restraint structure that acts to restrain movement of the guide wire when cover 384 is in the closed position. As shown, the restraint structure includes a plurality of tabs 396 extending from the lower surface of cover 384. The top surface of chassis 382 includes a plurality of recesses 398 configured to receive tabs 396 when cover 384 is in the closed position. Tabs 396 are positioned such that when cover 384 is closed, tabs 396 are positioned over guide wire channel 390 such that tabs 396 prevent guide wire 301 from falling out of guide wire channel 390 (i.e., restrains movement of the guide wire in a direction perpendicular to the top surface of chassis 382). In addition, the sidewalls of guide wire channel 390 and the engagement surfaces of wheels 522 and 524 prevent or restrain movement of guide wire 301 in other directions perpendicular to the longitudinal axis of guide wire 301. Thus, tabs 392 and guide wire channel 390 hold guide wire 301 within channel 390 during rotation of rotational drive assembly 326.

When cover 384 is in the open position, guide wire channel 390 is exposed allowing the user to load cassette 300 with a guide wire. With cover 384 open, guide wire 301 is loaded into rotational drive assembly 326 by placing the guide wire into guide wire channel 390. Tabs 392 facilitate the placement of guide wire 301 by aiding the user in aligning the guide wire with guide wire channel 390. As will be described in more detail below, once guide wire 301 is positioned within guide wire channel 390 engagement surfaces of engagement structure 386 are brought into engagement with the guide wire. In one embodiment, when the user activates controls (e.g., controls 16 located at workstation 14) to open cover 384, rotational drive assembly 326 is automatically rotated such that guide wire channel 390 is facing generally upward to allow for easy loading or removal of guide wire 301.

In one embodiment, cassette 300 is a modular cassette that allows various components of cassette 300 to be removed and/or switched out with other components. In an exemplary embodiment, a user may wish to control the guide wire using bedside system 12 and to control the working catheter manually. In this embodiment, a user may mount only guide wire axial drive mechanism 350 and rotational drive assembly 326 within housing 316 of cassette 300. In another exemplary embodiment, a user may wish to control the working catheter using bedside system 12 and to control the guide wire manually. In this embodiment, a user may mount only working catheter drive mechanism 352 within housing 316 of cassette 300. In another embodiment, cassette 300 may include additional locations for mounting drive mechanisms for any type of additional catheter devices that may be used during a procedure. For example, a user may be able to couple drive mechanisms to cassette 300 to control the movement and/or control of an intravascular ultrasound catheter.

Referring to FIG. 9, cassette 300 is shown in the “loaded” or “use” position. In the “loaded” position, y-connector support assembly 322 is rotated downward such that y-connector 338 is aligned with guide wire channel 364 of axial drive assembly 324. The axial alignment allows guide wire 301 and working catheter 303 to be moved into and/or out of y-connector 338 via operation of guide wire axial drive mechanism 350 and working catheter axial drive mechanism 352. Cover 356 is shown in the closed position overlying both the guide wire axial drive mechanism 350 and the working catheter axial drive mechanism 352. As shown, cover 356 also covers guide wire channel 364, working catheter drive channel 366 and secondary channel 650. As such, cover 356 acts to prevent interference with the various components of axial drive assembly 324 during use.

During use of cassette 300 to perform a catheter based medical procedure, guide wire 301 and working catheter 303 are moved into the patient's body (typically, into an artery of the patient) and various fluids (e.g., contrast agent, medicine, etc.) may be delivered into the patient via the guide catheter. Thus, during a procedure, guide wire 301 and working catheter 303 typically will come into contact with bodily fluids (e.g., blood) or other fluids (e.g., contrast agent) administered to the patient during the procedure. In one embodiment, cassette 300 is equipped with a structure configured to remove fluid from the outer surfaces of guide wire 301 and working catheter 303 as the guide wire or catheter is retracted from the patient and back into cassette 300. Such a structure decreases the amount of fluid that remains on the guide wire and working catheter as they come into contact with the wheels of the various drive assemblies. Because the presence of fluid on the outer surface of the guide wire or catheter may impact the transmission of motion from the drive assemblies to the devices, limiting or preventing the amount of fluid that remains on the devices as they enter cassette 300 may improve the performance of cassette 300.

In one embodiment, the proximal end of y-connector 338 may include a ring element 662 that includes an inner surface that is in contact with the outer surface of guide wire 301 and working catheter 303. The inner surface of ring element 662 acts to wipe fluid from the outer surface of guide wire 301 and working catheter 303 as the devices are retracted back into cassette 300. In one embodiment, the inner surface of ring element 662 may be formed of a compliant, rubber-like polymer material that pushes or scrapes fluid from the outer surfaces of the devices as the devices are drawn past the surface of ring element 662. In various other embodiments, the fluid removing ring element 662 may be coupled to the outer surface of top deck 354 and may be located at the front of guide wire channel 364. In another embodiment, fluid removing ring element 662 may be located within cassette 300 in front of the guide wire and working catheter axial drive mechanisms. In another embodiment, cassette 300 may include a first ring element located within guide wire channel 364 configured to remove or wipe fluid from guide wire 301 and a second ring element located within working catheter drive channel 366 configured to remove or wipe fluid from working catheter 303.

After cover 356 is moved to the closed position, handle 358 is rotated approximately 90 degrees such that a portion of handle 358 is positioned over cover 356. As will be discussed in greater detail below, rotation of handle 358 to the closed position shown in FIG. 9 causes the engagement surface of the guide wire axial drive mechanism 350 and of the working catheter axial drive mechanism 352 to move together engaging the guide wire and working catheter, respectively.

In addition, when cassette 300 is moved to the “loaded” position, cover 384 is moved to the closed position overlying rotational drive mechanism 380 and guide wire channel 390 as shown in FIG. 9. Like cover 356, cover 384 acts to prevent interference with the various components of rotational drive assembly 326 during use. In one embodiment, a user may activate controls (e.g., controls located at workstation 14) to cause the various components of cassette 300 to move between the “loading” and “loaded” positions. In addition, cassette 300 may also be configured to allow the user to move the various components of cassette 300 between the “loading” and “loaded” positions manually.

Referring to FIG. 9, in the “loaded” or “use” configuration, the longitudinal axis (and the internal lumen) of y-connector 338 is aligned with guide wire channel 364 of axial drive assembly and with guide wire channel 390 of rotational drive assembly 326. This alignment provides a path extending from the rear of cassette 300 through y-connector 338 into the guide catheter through which the guide wire is advanced or retracted during axial movement of the guide wire. In various embodiments, components of cassette 300, including top deck 354, chassis 382, cover 356, and cover 384, may be made from a transparent or translucent plastic.

Some procedures may be performed using more than one working catheter (e.g., first working catheter 303 and second working catheter 660). As shown in FIG. 9, during such a procedure, a second working catheter 660 may be positioned within secondary channel 650 while first working catheter 303 is positioned within working catheter drive channel 366. For these procedures, secondary channel 650 provides a storage or holding location for a second working catheter while the first working catheter is engaged within working catheter drive channel 366. Thus, secondary channel 650 holds the second working catheter while the user is manipulating the first working catheter with cassette 300. When the user wants to control second working catheter 660 using cassette 300, cover 356 is moved to the open position. Second working catheter 660 is then moved from secondary channel 650 to the working catheter drive channel 366, and first working catheter 303 is moved from working catheter drive channel 366 to secondary channel 650. Cover 356 is then closed causing the second working catheter to be engaged within working catheter drive channel 366 to allow the user to control second working catheter 660 via cassette 300.

Referring to FIG. 10, an exploded perspective view from above of axial drive assembly 324 is shown. FIG. 10 generally depicts the components of axial drive assembly 324. Guide wire axial drive mechanism 350 and working catheter axial drive mechanism 352 are positioned above base plate 318, and top deck 354 is fastened to central portion 360 of base plate 318 above guide wire axial drive mechanism 350 and working catheter axial drive mechanism 352. Thus, guide wire axial drive mechanism 350 and working catheter axial drive mechanism 352 are generally enclosed within a chamber defined by top deck 354 and central portion 360 of base plate 318 when axial drive assembly 324 is assembled. Top deck 354 includes a plurality of apertures 362 to receive various portions of both axial drive mechanism 350 and working catheter axial drive mechanism 352.

Axial drive mechanism 350 includes a drive element 400, a first roller assembly 402, a second roller assembly 404, and a guide wire axial motion sensor assembly, shown as encoder assembly 406. First roller assembly 402 and second roller assembly 404 are both mounted within a housing 416. Drive element 400 includes a drive shaft 408, a drive wheel 410, a bearing 412, and a screw 414. Drive shaft 408 is configured to engage second capstan 306 of motor drive base 302 such that drive shaft 408 and drive wheel 410 rotate in response to rotation of second capstan 306. First roller assembly 402 includes an idler wheel or roller 418, a wheel housing 420, a bearing 422, and a spring 424.

Drive wheel 410 includes an outer or engagement surface 426, and roller 418 includes an outer or engagement surface 428. Generally, when guide wire axial drive mechanism 350 is placed in the “use” or “engaged” position (shown in FIG. 13), guide wire 301 is positioned between drive wheel 410 and roller 418 such that engagement surface 426 of drive wheel 410 and engagement surface 428 of roller 418 are able to engage the guide wire. In this embodiment, engagement surface 426 and engagement surface 428 define a pair of engagement surfaces. The force applied to guide wire 301 by engagement surface 426 and engagement surface 428 is such that drive wheel 410 is able to impart axial motion to guide wire 301 in response to the rotation of drive shaft 408 caused by rotation of second capstan 306. This axial motion allows a user to advance and/or retract a guide wire via manipulation of controls 16 located at workstation 14. Roller 418 is rotatably mounted within wheel housing 420 and rotates freely as drive wheel 410 rotates to drive guide wire 301. Spring 424 is biased to exert a force onto wheel housing 420 causing roller 418 to engage the guide wire against drive wheel 410. Spring 424 is selected, tuned, and/or adjusted such that the proper amount of force is applied to guide wire 301 by engagement surface 426 and engagement surface 428 in the “engaged” position. In other embodiments, additional drive elements may be added as necessary to impart axial motion to the guide wire.

Second roller assembly 404 includes an idler wheel or roller 430, a wheel housing 432, a bearing 434, and a spring 436. Encoder assembly 406 includes shaft 438, magnetic coupling 440, idler wheel or roller 442, bearing 444, and a screw 446. Roller 430 includes an outer or engagement surface 448 and roller 442 includes an outer or engagement surface 450.

In the “engaged” position, guide wire 301 is positioned between roller 430 and roller 442 such that engagement surface 448 of roller 430 and engagement surface 450 of roller 442 are able to engage the guide wire. In this embodiment, engagement surface 448 and engagement surface 450 define a pair of engagement surfaces. The force applied to guide wire 301 by engagement surface 448 and engagement surface 450 is such that drive wheel 410 is able to pull guide wire 301 past roller 430 and 442. In this way, the pair of non-active or idle rollers 430 and 442 help support guide wire 301 and maintain alignment of guide wire 301 along the longitudinal axis of cassette 300.

Roller 430 is rotatably mounted within wheel housing 432, and roller 442 is rotatably mounted to shaft 438. Both rollers 430 and 442 are mounted to rotate freely as drive wheel 410 imparts axial motion to guide wire 301. Spring 436 is biased to exert a force onto wheel housing 432 causing roller 430 to engage guide wire 301 against roller 442. Spring 436 is selected, tuned, and/or adjusted such that the proper amount of force is applied to guide wire 301 by engagement surface 448 and engagement surface 450 in the “engaged” position to support the guide wire while still allowing the guide wire to be moved axially by drive wheel 410. In other embodiments, additional pairs of non-active or idler rollers may be added as needed to provide proper support and alignment for the guide wire. In one embodiment, spring 424 and spring 436 are selected or adjusted such that the force applied to guide wire 301 by wheels 430 and 442 is approximately the same as the force applied to guide wire 301 by wheels 410 and 418.

Encoder assembly 406 includes magnetic coupling 440 that engages a magnetic encoder located within motor drive base 302. The magnetic encoder is configured to measure an aspect (e.g., speed, position, acceleration, etc.) of axial movement of the guide wire. As roller 442 rotates, shaft 438 rotates causing magnetic coupling 440 to rotate. The rotation of magnetic coupling 440 causes rotation of the magnetic encoder within motor drive base 302. Because rotation of roller 442 is related to the axial movement of guide wire 301, the magnetic encoder within motor drive base 302 is able to provide a measurement of the amount of axial movement experienced by guide wire 301 during a procedure. This information may be used for a variety of purposes. For example, this information may be displayed to a user at workstation 14, may be used in a calculation of or estimated position of the guide wire within the vascular system of a patient, may trigger an alert or alarm indicating a problem with guide wire advancement, etc.

As shown in FIG. 10, first roller assembly 402 and second roller assembly 404 are both mounted within a housing 416. Housing 416 provides a common support for first roller assembly 402 and second roller assembly 404. As will be discussed in more detail below, first roller assembly 402 and second roller assembly 404 are moved away from drive wheel 410 and roller 442, respectively, when axial drive assembly 324 is placed in the “loading” configuration. This facilitates placement of guide wire 301 between the opposing pairs of engagement surfaces of guide wire axial drive mechanism 350. Housing 416 allows first roller assembly 402 and second roller assembly 404 to be moved together (e.g., in sync) away from drive wheel 410 and roller 442, respectively, when axial drive assembly 324 is placed in the “load” configuration.

Axial drive assembly 324 also includes working catheter axial drive mechanism 352. Working catheter axial drive mechanism 352 includes a drive element 452 and a working catheter axial motion sensor assembly, shown as working catheter encoder assembly 454. Drive element 452 includes a drive shaft 456, a drive wheel 458, a bearing 460, and a screw 462. Drive shaft 456 is configured to engage first capstan 304 of motor drive base 302 such that drive shaft 456 and drive wheel 458 rotate in response to rotation of first capstan 304. Encoder assembly 454 includes shaft 464, a roller 466, an encoder linkage 468, a spring 470, and a magnetic coupling 480.

Drive wheel 458 includes an outer or engagement surface 472 and roller 466 includes an outer or engagement surface 474. When working catheter axial drive mechanism 352 is in the “engaged” position, a working catheter is positioned between drive wheel 458 and roller 466, such that engagement surface 472 and engagement surface 474 are able to engage working catheter 303. In this embodiment, engagement surfaces 472 and 474 define a pair of engagement surfaces. The force applied to working catheter 303 by engagement surfaces 472 and 474 is such that drive wheel 458 is able to impart axial motion to the working catheter in response to the rotation of drive shaft 456 caused by rotation of first capstan 304. This axial motion allows a user to advance and/or retract a working catheter via manipulation of controls located at workstation 14. Roller 466 is rotatably mounted to shaft 464 and rotates freely as drive wheel 458 rotates to drive the working catheter.

Spring 470 is coupled to a first end of linkage 468. The second end of linkage 468 includes an aperture 476 that is pivotally coupled to a post 478 extending from the inner surface of top deck 354. Spring 470 is biased to exert a force on to linkage 468 causing linkage 468 to pivot about post 478 to force roller 466 to engage working catheter 303 against drive wheel 458. Spring 470 is selected, tuned, and/or adjusted such that the proper amount of force is applied to working catheter 303 by engagement surfaces 472 and 474 in the “engaged” position to allow drive wheel 458 to impart axial movement to the working catheter.

Encoder assembly 454 includes magnetic coupling 480 that engages a magnetic encoder located within motor drive base 302. The magnetic encoder is configured to measure an aspect (e.g., speed, position, acceleration, etc.) of axial movement of the working catheter. As roller 466 rotates, shaft 464 rotates causing magnetic coupling 480 to rotate. The rotation of magnetic coupling 480 causes rotation of the magnetic encoder within motor drive base 302. Because rotation of roller 466 is related to the axial movement of working catheter 303, the magnetic encoder within motor drive base 302 is able to provide a measurement of the amount of axial movement experienced by the working catheter during a procedure. This information may be used for a variety of purposes. For example, this information may be displayed to a user at workstation 14, may be used in a calculation of or estimated position of the working catheter within the vascular system of a patient, may trigger an alert or alarm indicating a problem with working catheter advancement, etc.

As will be discussed in more detail below, roller 466 is moved away from drive wheel 458 when axial drive assembly 324 is placed in the “loading” configuration. This facilitates placement of the working catheter between the opposing pairs of engagement surfaces of working catheter axial drive mechanism 352.

In one embodiment, cassette 300 and/or motor drive base 302 includes a locking mechanism that is configured to lock the position of guide wire 301 during manipulation of the working catheter 303 and to lock the position of working catheter 303 during manipulation of guide wire 301. In one embodiment, the locking mechanism acts to increase the force applied to the guide wire by the engagement surfaces when the working catheter is being advanced and to increase the force applied to the working catheter by the engagement surfaces when the guide wire is being advanced.

Referring to FIGS. 10 and 11, top deck 354 includes a plurality of cylindrical sleeves, first sleeve 482, second sleeve 484, and third sleeve 486, extending from the inner or lower surface of top deck 354. Top deck 354 also includes a plurality of cylindrical collars, first collar 488, second collar 490, and third collar 492, extending from the upper surface of top deck 354. Collar 488 is in axial alignment with sleeve 482. Collar 490 is in axial alignment with sleeve 484. Collar 492 is in axial alignment with sleeve 486. Each of the collars 488, 490, and 492 define an aperture 362. In the embodiment shown, sleeve 482 and collar 488 are configured to receive working catheter drive element 452, sleeve 484 and collar 490 are configured to receive guide wire drive element 400, and sleeve 486 and collar 492 are configured to receive guide wire encoder assembly 406. Apertures 362 provide access to screws 414, 446, and 462 once top deck 354 is mounted over axial drive assembly 324.

Top deck 354 includes a collar 494 aligned with and located at the back end of guide wire channel 364. Collar 494 is configured to receive front shaft 512 that extends from chassis 382 of rotational drive assembly 326. Collar 494 is configured to allow front shaft 512 (and consequently the rest of rotational drive assembly 326) to rotate about the longitudinal axis of guide wire channel 390 relative to axial drive assembly 324. In one embodiment, rotational drive assembly 326 is able to rotate relative to housing 316 of cassette 300 while axial drive assembly 324 does not rotate relative to housing 316. In another embodiment, both rotational drive assembly 326 and axial drive assembly 324 rotate relative to housing 316 of cassette 300.

FIG. 11 is a bottom perspective view of cassette 300 showing top deck 354 mounted above guide wire axial drive mechanism 350 and working catheter axial drive mechanism 352. FIG. 11 shows working catheter drive element 452, guide wire drive element 400, and guide wire encoder assembly 406 received within sleeves 482, 484, and 486. A support structure 496 extends from the lower surface of top deck 354. Spring 470 is coupled at one end to support structure 496 allowing spring 470 to compress and expanded between linkage 468 and support structure 496.

As shown, the lower end of drive shaft 408 includes a keyed recess 498, and the lower end of drive shaft 456 includes a keyed recess 500. Keyed recess 500 is one embodiment of first capstan socket 310, and keyed recess 498 is one embodiment of second capstan socket 312. Keyed recess 500 is configured to receive a capstan, such as first capstan 304, and keyed recess 498 is configured to receive a capstan, such as second capstan 306. First capstan 304 and second capstan 306 are keyed to fit within keyed recess 500 and 498 and to engage and turn drive shafts 456 and 408 upon rotation of the capstans.

As shown, magnetic coupling 440 of guide wire encoder assembly 406 includes a circular array of magnets 504. Magnetic coupling 480 of working catheter encoder assembly 454 includes a circular array of magnets 506. Magnetic couplings 440 and 480 engage with magnetic encoders positioned within motor drive base 302. The magnetic encoders of motor drive base 302 are coupled to appropriate electronics to detect and measure rotation of rollers 442 and 466 and to calculate axial motion of guide wire 301 and working catheter 303 based on the measured rotations. While this embodiment discloses the use of magnetic encoders to detect the axial motion of the guide wire and working catheter, other sensors may be used. In one embodiment, axial motion of the guide wire may be detected by an optical sensor that detects movement of the guide wire and/or working catheter by scanning the surface of the guide wire and/or working catheter as it passes the optical sensor. In one such embodiment, the optical sensor includes an LED light source and a detector (e.g., a complimentary metal oxide semiconductor, other light detecting circuitry, etc.) that detects light reflected off the surface of the guide wire and/or working catheter, and the light detected by the detector is analyzed (e.g., by a digital signal processor) to determine movement of the guide wire and/or working catheter. In another embodiment, the surface of the guide wire and/or working catheter may include indicia that are detected to determine axial movement of the guide wire. In other embodiments, other types of sensors (e.g., resolvers, sychros, potentiometers, etc.), may be used to detect movement of the guide wire and/or working catheter.

Cassette 300 also includes a series of magnets 508 positioned below guide wire channel 364. Because, in at least some embodiments, the guide wire is made from a magnetic material, magnets 508 are able to interact with the guide wire. In this embodiment, the magnetic attraction created by magnets 508 helps the user position guide wire 301 during loading by drawing guide wire 301 into guide wire channel 364. The magnetic attraction created by magnets 508 also tends to hold guide wire 301 within guide wire channel 364 during advancement and/or retraction of the guide wire. Further, magnets 508 help to hold guide wire 301 straight (i.e., parallel to the longitudinal axis of guide wire channel 364) to aid in the axial movement caused by guide wire axial drive mechanism 350.

FIG. 12 shows a top view of axial drive assembly 324 in the “loading” configuration with handle 358 (shown in broken lines) rotated such that handle 358 is generally parallel to guide wire channel 364. FIG. 13 shows a top view of axial drive assembly 324 in the “loaded” or “use” configuration with handle 358 rotated such that it is generally perpendicular to guide wire channel 364. Generally, when handle 358 is moved from the position of FIG. 13 to the position of FIG. 12, the engagement surfaces of both guide wire axial drive mechanism 350 and working catheter axial drive mechanism 352 are moved away from each other increasing the space between the pairs of wheels in the drive mechanisms. This provides sufficient space between the wheels of each drive mechanism to allow the user to place guide wire 301 and working catheter 303 into the channels between the wheels. Generally, as handle 358 is moved from the position of FIG. 12 to the position of FIG. 13, the engagement surfaces of both guide wire axial drive mechanism 350 and working catheter axial drive mechanism 352 are moved toward each other bringing the engagement surfaces of each drive mechanism into engagement with guide wire 301 and working catheter 303, respectively.

In the embodiment shown, handle 358 is coupled to a shaft 357. Shaft 357 includes a cam section 359 and housing 416 includes a cam surface 417. As handle 358 rotates from the position shown in FIG. 12 to the position shown in FIG. 13, cam section 359 of shaft 357 moves along cam surface 417 causing housing 416 to move toward guide wire 301. This motion engages guide wire 301 between drive wheel 410 and roller 418 and between roller 430 and roller 442. When handle 358 is brought into the position of FIG. 13, springs 424 and 436 are compressed to the proper tension to allow drive wheel 410 to move guide wire 301 axial along its longitudinal axis.

In addition, housing 416 includes a tab 419 that is coupled to linkage 468. Thus, linkage 468 rotates about post 478 when housing 416 is moved to the position shown in FIG. 12. This movement draws roller 466 away from working catheter drive wheel 458. When, housing 416 is moved to the position shown in FIG. 13, roller 466 is moved toward catheter drive wheel 458 such that the engagement surfaces of roller 466 and drive wheel 458 engage working catheter 303. In one embodiment, cassette 300 is configured to allow the user to move the axial drive assembly 324 between the “use” and “loading” positions via manipulation of controls at workstation 14. Cassette 300 may also be configured to allow the user to move the axial drive assembly 324 between the “use” and “loading” position manually.

FIGS. 14 and 15 show a perspective view of rotational drive assembly 326 showing cover 384 in the open position. Rotational drive assembly 326 includes rotational drive mechanism 380, chassis 382, an engagement structure 386, and a disengagement assembly 510. Chassis 382 fits over engagement structure 386 and provides mounting for various components of rotational drive assembly 326. Chassis 382 includes a front shaft 512 and a rear shaft 514. As discussed above, front shaft 512 is rotatably received within collar 494 of top deck 354, and rear shaft 514 is rotatably received within collar 516 such that rotational drive mechanism 380 is able to rotate relative to journal 388. As shown, collar 516 extends through and is supported by journal 388 such that rear shaft 514 rotates within collar 516 as rotational drive mechanism 380 is rotated. Collar 516 rests within a recess or slot formed within journal 388. In another embodiment, rear shaft 514 may be in direct contact with journal 388 such that rear shaft 514 rotates within the recess or slot of journal 388 as rotational drive mechanism 380 is rotated. Guide wire channel 390 extends the length of chassis 382 through both front shaft 512 and rear shaft 514.

Rotational drive mechanism 380 includes rotation bevel gear 518 that engages a drive gear 520. Bevel gear 518 is rigidly coupled to front shaft 512 of chassis 382 such that rotation of bevel gear 518 rotates chassis 382. Drive gear 520 is coupled to a rotational actuator positioned in motor drive base 302 and engages bevel gear 518. Rotation of the rotational actuator in motor drive base 302 causes drive gear 520 to rotate which causes bevel gear 518 to rotate which in turn causes rotational drive mechanism 380 to rotate. Rotational drive mechanism 380 is allowed to rotate about the longitudinal axis of guide wire channel 390 via the rotatable connections between front shaft 512 and top deck 354 and between rear shaft 514 and journal 388. Bevel gear 518 further includes a slot 519 in axial alignment with guide wire channel 390. Slot 519 allows the user to place guide wire 301 into guide wire channel 390 by dropping it in vertically as opposed to threading it through bevel gear 518. In one embodiment, rotational drive assembly 326 is equipped with one or more sensors that are configured to measure an aspect (e.g., speed, position, acceleration, etc.) of rotation of the guide wire and/or any other structure of rotational drive assembly 326. The sensors that measure rotation of the guide wire may include magnetic encoders and/or optical sensors as discussed above regarding the sensors that measure axial motion of the guide wire and/or working catheter. However, any suitable sensor (e.g., resolvers, sychros, potentiometers, etc.) may be used to detect rotation of the guide wire.

Referring to FIG. 15, engagement structure 386 is shown according to an exemplary embodiment. As shown, engagement structure 386 includes four pairs of idler wheels or rollers. Each pair of rollers includes a fixed wheel 522 and an engagement wheel 524. Fixed wheels 522 are rotatably coupled to chassis 382 via fixation posts 530. Each engagement wheel 524 is part of an engagement wheel assembly 523. Each engagement wheel assembly 523 includes a pivot yoke 532 and a spring 536. Each engagement wheel is mounted to pivot yoke 532 via a mounting post 538. Each pivot yoke 532 is pivotally coupled to chassis 382 via fixation posts 534.

Each fixed wheel 522 includes an outer or engagement surface 526 and each engagement wheel 524 includes an outer or engagement surface 528. Generally, FIG. 14 shows engagement structure 386 in the “use” or “engaged” position. In the “engaged” position, guide wire 301 is positioned between fixed wheels 522 and engagement wheels 524 such that engagement surfaces 526 and 528 are able to engage guide wire 301. In this embodiment, engagement surface 526 and engagement surface 528 of each pair of rollers define a pair of engagement surfaces. The force applied to guide wire 301 by engagement surfaces 526 and 528 is sufficient to cause the guide wire to rotate about its longitudinal axis as rotational drive assembly 326 is rotated. Further, the force applied to guide wire 301 by engagement surfaces 526 and 528 is also sufficient to allow the guide wire to be moved axially by guide wire axial drive mechanism 350.

Springs 536 are biased to exert a force onto pivot yokes 532 causing each engagement wheel 524 to engage the opposite fixed wheel 522. The generally L-shape of pivot yoke 532 allows springs 536 to be aligned with the longitudinal axis of guide wire 301 and still cause engagement between engagement wheels 524, fixed wheels 522, and the guide wire. This allows the lateral dimension of rotational drive assembly 326 to be less than if springs 536 were positioned perpendicular to the longitudinal axis of the guide wire. Springs 536 are selected, tuned, and/or adjusted such that the proper amount of force is applied to the guide wire by engagement surfaces 526 and 528 in the “engaged” position.

Cassette 300 also includes a series of magnets 540 located beneath guide wire channel 390. Because, in at least some embodiments the guide wire is made from a magnetic material, magnets 540 are able to interact with the guide wire. In this embodiment, the magnetic attraction created by magnets 540 helps the user position guide wire 301 during loading by drawing guide wire 301 into guide wire channel 390. The magnetic attraction created by magnets 540 also tends to hold guide wire 301 within guide wire channel 390 during advancement and/or retraction of the guide wire. Further, magnets 540 help to hold guide wire 301 straight (i.e., parallel to the longitudinal axis of guide wire channel 390) to aid in the axial movement caused by guide wire axial drive mechanism 350.

Rotational drive assembly also includes a disengagement assembly 510. Disengagement assembly 510 includes a stepped collar 542, a base plate 544, and a spring 546. Stepped collar 542 is coupled to base plate 544, and spring 546 is coupled at one end to chassis 382 and at the other end to base plate 544. Stepped collar 542 includes a slot 548 in axial alignment with guide wire channel 390. Like slot 519, slot 548 allows the user to place guide wire 301 into guide wire channel 390 by dropping it in vertically as opposed to threading it through stepped collar 542. Base plate 544 includes a plurality of engagement arms 550 that extend generally perpendicular to the plane defined by base plate 544.

Generally, disengagement assembly 510 allows engagement wheels 524 to be moved away from fixed wheels 522. Referring to FIGS. 16 and 17, FIG. 17 shows a top view of rotational drive assembly 326 in the “loading” configuration, and FIG. 16 shows a top view of rotational drive assembly 326 in the “loaded” or “use” configuration. To cause engagement wheels 524 to disengage from guide wire 301, an axially directed force (depicted by the arrow in FIG. 17) is applied to stepped collar 542. This causes base plate 544 to move toward the front of cassette 300 in the direction of the arrow. As base plate 544 moves forward, spring 546 is compressed, and engagement arms 550 are brought into contact with pivot yokes 532. The contact between engagement arms 550 and pivot yokes 532 causes springs 536 to be compressed, and pivot yokes 532 pivot about fixation posts 534. As pivot yokes 532 pivot, engagement wheels 524 are drawn away from fixed wheels 522. As shown in FIG. 17, this provides sufficient space between engagement wheels 524 and fixed wheels 522 to allow the user to place guide wire 301 into guide wire channel 390.

When the axial force is removed from stepped collar 542, engagement wheels 524 move from the position shown in FIG. 17 to the “engaged” position shown in FIG. 16. When the axial force is removed, spring 546 and springs 536 are allowed to expand causing engagement arms 550 to disengage from pivot yokes 532. Pivot yokes 532 pivot counter-clockwise about fixation posts 534, bringing engagement wheels 524 back toward guide wire channel 390 causing engagement surfaces 526 of fixed wheels 522 and engagement surfaces 528 of engagement wheels 524 to engage guide wire 301.

In one embodiment, a user may activate controls located at workstation 14 to cause rotational drive assembly 326 to move between the “use” position and the “loading” position. In this embodiment, rotational drive assembly 326 is automatically rotated such that guide wire channel 390 is facing generally upward to allow for easy loading or removal of the guide wire. In the embodiment shown, chassis 382 rotates relative to stepped collar 542. In this embodiment, when rotational drive assembly 326 is in the “loading” position, a path defined by the engagement surfaces of engagement structure 386 and guide wire channel 390 align with slot 548 of stepped collar 542. Motor drive base 302 may also include a structure (e.g., two rods, etc.) that applies the axial force to stepped collar 542 in response to a user's activation of controls located at workstation 14. The structure applies the axial force to the stepped collar 542 to cause engagement structure 386 to disengage from the guide wire. Next, cover 384 is moved from the closed position to the open position allowing the user to access guide wire channel 390 to either remove or install the guide wire. In one embodiment, cassette 300 and/or motor drive base 302 includes motors or other actuators that cause the covers of cassette 300 to open in response to a user's activation of controls at workstation 14.

In various embodiments, cassette 300 may be configured to facilitate transfer or replacement of a guide wire during a catheter procedure. Referring to FIG. 18, a rear perspective view of cassette 300 with outer cassette cover 320 attached is shown, according to an exemplary embodiment. In an exemplary embodiment, cassette 300 includes a secondary support assembly, shown as guide wire support structure 670, coupled to and extending above the upper edge of journal 388. Support structure 670 provides a storage or holding location to hold a guide wire while a user either loads a different guide wire into cassette 300 or removes a different guide wire from cassette 300. In this manner, support structure 670 provides a convenient location to place one guide wire while the user of the cassette is occupied with adding or removing another guide wire from cassette 300.

Support structure 670 includes an outer housing 672 and an insert 674 positioned within outer housing 672. Together, outer housing 672 and insert 674 are shaped to define a channel 676 configured to receive a guide wire. As shown, the upper portions of outer housing 672 and insert 674 are angled defining an angled, “V-shaped” upper section 680 of channel 676, and the lower portions of outer housing 672 and insert 674 are shaped defining a lower, vertically oriented slot 678. A guide wire may be placed into and supported within channel 676, while the user handles a second guide wire. In the embodiment shown, the upper angled section 680 of channel 676 helps guide the guide wire into channel 676, and the guide wire is held within slot 678. In one embodiment, insert 674 may be made from a compliant material (e.g., a polymer material, rubber, etc.) that helps grip the guide wire without damaging or altering the outer surface of the guide wire.

Referring to FIGS. 19-22, a catheter force measurement apparatus 700 includes a housing 702 through which a portion of a guide wire or working catheter extends. The catheter force measurement apparatus 700 will be described in connection with a guide wire 704. However, force measurement apparatus 700 could be used with a working catheter as well. A guide wires is typically formed of flexible material. The guide wire is translated along its longitudinal axis to a location of interest such as an occlusion in a lumen of a patient. The distal free end of a guide wire 704 that is located within a lumen of a patient may abut against the wall of the lumen or abut against an occlusion or a juncture in the vascular system as the free end of the guide wire is transitioning from one lumen to another branch of the vascular system. Since guide wire 704 is an elongated flexible member, a portion of guide wire 704 may begin to buckle and fold upon itself if the free end has hit an obstruction and the guide wire is continued to be translated into the patient. If a free end of guide wire 704 is unable to proceed due to some obstruction, and an operator such as a physician continues to translate the guide wire into the vasculature, a portion of the guide wire 704 may buckle and/or folds on itself. This issue may not be detected by a physician until an x-ray or other image is taken of the patient.

Catheter force measurement apparatus 700 may be incorporated into an existing robotic catheter system such as the catheter system shown in FIGS. 1-18 discussed above. Referring to FIG. 19, catheter force measurement apparatus 700 may be included as an integral part of a cassette. In another embodiment catheter force measurement apparatus 700 is positioned immediately after a translation module that applies a force to the guide wire in the direction along its longitudinal axis. Both locations are or may be included as a stand alone module outside the cassette. Both possible locations are illustrated in FIG. 19 in dashed lines. Catheter force measurement apparatus 700 includes a housing 702 that has a track having a curved portion 706 having an inner supporting wall 708 and defining an outer region 710. An idler wheel 712 is located proximate inner supporting wall 708. Idler wheel 712 is includes a wheel member 714 having an engagement surface 716. Wheel member 714 rotates about its center axis 718.

In one embodiment, wheel member 714 is secured to an axle 720 through center axis 718 which extends vertically and perpendicular to a horizontal surface 722 of the housing. Axle 720 extends below surface 722. Axle 720 includes a first end proximate the idler wheel 712 and an opposing distal end 724. Axle 720 is pivotally secured to a pivot 726 below surface 722 to permit idler wheel 712 to move away from inner supporting wall 708 into open region 710. A plate 728 is secured to axle 720 proximate to distal end 724. Movement of the plate 24 is then sensed by sensor 730 such as an optical sensor or other type of sensor as known in the art such as magnetic sensor or electro mechanical and mechanical type sensor that can detect movement of the distal end 724 of axle 720. Referring to FIG. 21 a spring 732 or other biasing member biases axle 720 in a first vertical position such that engagement surface 716 of wheel member 714 is located proximate inner supporting wall 708. When guide wire 704 moves toward open region 710 wheel 714 is pushed away from curved supporting wall 708. This causes axle 720 to pivot about pivot 26 resulting in movement of plate 728 which in turn is sensed by sensor 730.

Catheter force measurement apparatus 700 may be used in a catheter drive mechanism as described herein. Referring to FIG. 19 a first linear drive mechanism 350 includes a pair of drive wheels and a pair of matching idler wheels. A guide wire 704 is located between the drive wheels and idler wheels. As the drive wheels of the linear drive mechanism 350 are rotated about their respective axis, guide wire 704 is translated along its longitudinal axis in a fore/aft direction to insert and withdraw respectively a free end of the guide wire into the vasculature of a patient. As discussed above, a guide wire channel 364 guides the guide wire 704 as it is translated through the first linear drive mechanism. The features of catheter force measurement apparatus 700 may be incorporated into the cassette. Guide wire 704 as it exits channel 364 could enter catheter force measurement apparatus 700. In one embodiment catheter force measurement apparatus 700 may be positioned between the first drive mechanism 350 and working catheter axial drive mechanism 352. Guide wire 704 is positioned in the track of catheter measurement force apparatus 700. In one embodiment Guide wire 704 is urged against supporting wall 708 of curved portion 706 with idler wheel 712 and optional idler wheels or transition surfaces 740. The distance between idler wheels 740 and the radius of the curved portion 706 are sufficient to permit guide wire 704 to extend outwardly in response to a force on the free end and/or along the length of the guide wire. In another embodiment, guide wire 704 exits first drive mechanism 350 and is located in a first linear guide channel 742 maintaining guide wire in a straight orientation. Similarly, housing 702 may have a second exit channel 746 that maintains guide wire in a straight orientation that may be co-linear guide wire portion in the channel in the first linear guide channel immediately after the first drive mechanism 350. Transition regions 744 provide a transition of the guide wire from co-linear guides 742 and 746 to the curved portion 706. This alignment helps to maintain a portion of the guide wire against the supporting wall 708 of curved portion 706 during normal operation and translation of the guide wire.

Curved portion 706 only includes one supporting wall 708, the guide wire is free to move in an outwardly direction from supporting wall 706 toward open region 710. When the guide wire experiences a force on the tip and/or along the length of the guide wire within the lumen of a patient, the guide wire will naturally move out of alignment at the point where the guide wire is not in a straight line. This will occur at the curved portion 706. By allowing the guide wire to move out of alignment at a predictable point along its length it is possible to both control and measure this movement. The straight co-linear housing channels 742, 746 and s as well as the transition points 744 to the curved portion 706 provide a certain level of friction to avoid guide wire 704 from being moved out of position until a threshold force is applied to the free end or length of the guide wire.

As a force is applied to the free end of the guide wire or to a length of the guide wire within a lumen of a patient the guide wire has a tendency to buckle. The curved portion of the force measurement housing only supports the guide wire at the support wall. The guide wire is free to move outwardly from the support wall in to the open region. As a portion of the guide wire moves outwardly from the support wall idler wheel 712 is pushed in a direction of travel of the bulging or buckling portion of the guide wire. Referring to FIG. 22 as idler wheel 712 moves away from inner supporting wall 708, axle 720 is caused to pivot about pivot 726. Since the distance between idler wheel 712 and pivot 726 increases and decreases as idler wheel is moved away from and toward inner supporting wall 708, axle 720 must either be telescoping or permit for travel within idler wheel 712 such as within axle 718. Alternatively, idler wheel 712 may pivot as well about pivot 726 such that axle 720 is co-linear with axis 718. As axle 720 pivots, plate 728 is moved relative to sensor 730 and a signal may be sent to a controller remote from the catheter robotic system to an operator and/or physician. The movement provides an indication of the force being applied to the free end of the guide wire or along the length of the guide wire. In an alternative embodiment, sensor 730 may detect movement of a surface of idler wheel 712 as it move to and from inner support wall 706. Other sensors are also contemplated such as a sensor measuring the angle of rotation of pivot 726 that would correlate to the distance that idler wheel 712 moves and the amount of force being applied to guide wire 704.

The sensitivity of the idler wheel can be selected to allow the guide wire to move out of the curved portion in response to varying levels of force. For example, in certain types of procedure that are more sensitive, idler wheel can be adjusted such that very little force is required for the guide wire to buckle and trigger an event by the sensor.

The signal is processed by a processor such as a computer processor and if a certain level of motion is detected the processor then alerts the physician to the condition. The system then alerts the physician operator to the force condition by the interface either via a signal on a computer monitor, and/or a sound produced by the system, and/or via haptic feedback applied to the input device such as a joy stick that the physician is operating to translate the guide wire. The physician can then reverse movement of the guide wire by retracting the guide wire until the portion of the guide wire that was bulging or buckling in the force measurement housing reverts back to the support wall. As the guide wire returns to its original neutral operating position proximate the support wall the system will alert the physician that the force has been removed from the guide wire. Alternatively, when the physician is alerted that a force on the guide wire has exceeded a determined value for a particular procedure, the physician can take other action such as different maneuvers and manipulation of the guide wire to relive the force on the guide wire tip. For example, the physician may elect to rotate the guide wire to change the orientation of the tip of the guide wire prior to further translation of the guide wire. Alternatively, if tip of the guide wire is being held back from proceeding through the lumen by an occlusion, the physician may elect to repeatedly withdraw and advance in rapid succession to apply a force the free end of the guide wire through the occluded region.

Further modifications and alternative embodiments of various aspects of the invention will be apparent to those skilled in the art in view of this description. Accordingly, this description is to be construed as illustrative only. The construction and arrangements, shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The features described herein may be combined in any combination and such combinations are contemplated. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. A force measurement apparatus comprising:
a housing having a track with a curved guide wall having a convex shape configured to guide a portion of a guide wire;
a sensor proximate the first guide wall in a first position, the sensor sensing movement of a portion of the guide wire moving from a first position proximate the curved guide wall to a second position distal the first guide wall in a direction perpendicular to the longitudinal axis of the guide wire.

2. The force measurement apparatus of claim 1, wherein the housing includes an open region configured to permit a portion of the guide wire to move from the first guide wall to the open region.

3. The force measurement apparatus of claim 2, wherein the housing includes a first linear guide and a second linear guide co-linear with the first linear guide, the first guide wall being intermediate the first and second linear guides and not co-linear with the first and second linear guides.

4. The force measurement apparatus of claim 3, wherein the sensor includes an idler wheel with an engagement surface contacting a portion of the guide wire proximate the curved guide wall, the idler wheel movable in a direction away from the first guide wall.

5. The force measurement apparatus of claim 4, wherein the idler wheel is secured to an axle having a proximate and an opposing distal end, the axle being pivotally connected to a pivot intermediate the proximate and distal end, a sensor detects movement of the axle.

6. The force measurement apparatus of claim 5, wherein a spring member is secured to the axle to bias the idler wheel toward a first position.

7. The force measurement apparatus of claim 6, wherein the force applied by the spring to the has an adjustable force is adjustable.

8. A robotic catheter system, comprising:
a housing;
a linear drive mechanism supported by the housing and configured to engage and to impart linear movement to a guide wire along a longitudinal axis of the guide wire;
a track with a curved guide wall configured to guide a portion of the guide wire in an arcuate path and an open region allowing a portion of the guide wire to move into the open region in response to a force being applied to a free end of the guide wire;
a sensor proximate the first guide wall in a first position, the sensor sensing movement of a portion of the guide wire moving from a first position proximate the curved guide wall to a second position distal the first guide wall in a direction perpendicular to the longitudinal axis of the guide wire.

9. The robotic catheter system of claim 8, wherein the housing includes a first linear channel configured to guide the guide wire in a straight line, the first linear channel being located between the linear drive mechanism and the curved guide wall.

10. The robotic catheter system of claim 9, wherein the housing includes a second linear channel configured to guide the guide wire in a straight line, the curved guide wall being located intermediate the first and second linear channels.

11. The robotic catheter system of claim 10, wherein the first and second linear channels are co-linear.

12. The robotic catheter system of claim 11, the radius of the curved guide wall is sufficient to permit guide wire to extend outward from the curved guide wall in response to a force applied to the free end of the guide wire.

13. The robotic catheter system of claim 11, wherein the sensor includes a idler wheel movable away from the curved guide wall in response to movement of the guide wire away from the curved guide wall.

14. The robotic catheter system of claim 13, wherein the sensor includes a spring biasing the idler wheel toward the curved guide wall such that the guide wire is moved toward the curved guide wall when the force on the free end has been removed.

15. The robotic catheter system of claim 14, wherein the sensor includes an optical sensor that operatively tracks movement of the idler wheel, the optical sensor providing a signal to a controller to alert an operator when a predetermined threshold has been exceeded.

16. The robotic catheter system of claim 14, wherein the sensor includes an optical sensor that operatively tracks movement of the idler wheel, the optical sensor providing a signal to a controller to stop translation of the guide wire when a predetermined threshold distance of movement by the idler wheel has been met or exceeded.

17. A method for measuring the force applied to a guide wire comprising;
providing a channel having a first linear section with a wall on each side of the guide wire in a direction perpendicular to the movement of the guide wire;
providing a second curved convex section having a single wall and an open region in a direction perpendicular to the direction of travel;
permitting a portion of the guide wire to move from the curved convex section toward the open region in response to a force applied to a free end of the guide wire;
operatively connecting a sensor in the open region proximate the curved convex to measure movement of the guide wire away from the curved convex section toward the open region; and
providing a signal to a control station of the related to the amount movement of the guide wire from the a curved convex section toward the open region.

18. The method of claim 17, wherein the sensor includes an idler wheel biasing the guide wire toward the curved section.

19. The method of claim 18, wherein the curved convex section is intermediate the first linear section and a second linear section.

20. The cassette of claim 19, further providing a linear drive mechanism, where the curved convex portion is located between the linear drive mechanism and a patient.
